(19) United States
(12) Patent Application Publication
(10) Pub. No.: US 2005/0069939 A1
(43) Pub. Date: Mar. 31, 2005

(54) Amplification of polynucleotides by rolling circle amplification

(76) Inventors: Youxiang Wang, Palo Alto, CA (US); Yaping Zong, San Jose, CA (US)

Correspondence Address: RICHARD ARON OSMAN;SCIENCE AND TECHNOLOGY LAW GROUP, 242 AVE VISTA DEL OCEANO, SAN CLEMEMTE, CA, 92672, US

(21) Appl. No.: 10/952,076

(22) Filed: Sep. 27, 2004

Related U.S. Application Data
(60) Provisional application No. 60/506,218, filed on Sep. 26, 2003.

(51) Int. Cl. C12Q 1/68; C12P 19/34

(52) U.S. Cl. 435/6; 435/91.2

(57) ABSTRACT

Rolling circle amplification is used to amplify and detect target nucleic acid molecules by affixing a first and/or second linker nucleic acid molecule or a second linker nucleic acid molecule to the target nucleic acid molecule, then circularizing the target nucleic acid molecule, and then amplifying the circularized nucleic acid molecule to generate reiterated nucleic acid sequences. The methods may be used to amplify nucleic acids, particularly RNA, for detection and cloning.

### CROSS-REFERENCE TO RELATED APPLICATION

This application claims priority to U.S. Ser. No. 60/506,218, filed Sep. 26, 2003, entitled Amplification of Polynucleotide Sequences by Rolling Circle Amplification by inventors Youxiang Wang and Yaping Zong.

### FIELD OF THE INVENTION

The invention is in the field of methods of amplification of nucleic acids by rolling circle amplification (RCA).

### BACKGROUND

Obtaining a full-length cDNA is a critical, and often difficult task in characterizing genes. Traditional methods for cDNA library construction usually produce only partial cDNA fragments. Rapid amplification of cDNA ends (RACE) is one technique developed to recover full coding sequence; however, RACE technologies remain complicated and inefficient. Our invention using RT-RCA technology provides a vastly improved and simplified procedure for make full-length cDNA.

RT-PCR (reverse transcription-polymerase chain reaction) is a highly sensitive technique for mRNA detection and quantization. The technique consists of two parts: synthesis of cDNA from RNA by reverse transcription (RT) and amplification of a specific cDNA by polymerase chain reaction (PCR). Our invention using RT-RCA provides a vastly improved and simpler method of mRNA amplification and detection using rolling circle amplification.

Established nucleic acid amplification methods include methods based on cycling temperature such as PCR, LCR, and SPA, and methods using isothermal amplification such as NASBA, RCA, TMA, Q beta replicase and SDA. Various detection and amplification methods have recently been developed utilizing RCA; see, e.g. U.S. Pat. Nos. 5,871,921; 5,648,245; 5,866,377; 5,854,033; 6,287,824; 6,323,009.

### SUMMARY OF THE INVENTION

The invention provides methods of detection and cloning nucleic acid molecules that take advantage of rolling circle amplification. The invention provides methods of amplification, detection, and cloning of target nucleic acid molecules from complex mixtures using rolling circle amplification. The invention includes a number of advantages that may be found in various embodiments.

In one embodiment, the invention provides methods for circularizing entire target nucleic acid molecules for amplification. This allows cloning of mostly full-length target nucleic acid sequences and allows amplification and cloning of entire genomes if desired.

In another embodiment, the invention provides methods for amplifying and detecting desired region of target nucleic acid molecules using rolling circle amplification.

In another embodiment, the invention provides methods using a hairpin loop to create circular polynucleotides for rolling circle amplification and detection, instead of using padlock probes or an additional template for ligation to form circular nucleic acid molecules.

In another embodiment, the invention provides methods to circularize nucleic acid molecules using self-priming followed by closing the circle with ligation.

In another embodiment, the invention provides detection methods using the target sequence itself to generate a free 3′ end to initiate rolling circle amplification with circularized nucleic acid molecules, wherein the circularized nucleic acid molecules may comprise full-length gene sequences for gene specific detection and amplification, wherein addition of primers is not required. This allows detection without ligation, and few or no externally supplied primers for amplification, thus simplifying the overall reaction.

In another embodiment, the invention provides methods to generate a free 3′ end from the supplied fragments or oligonucleotides instead of the target for rolling circle amplification with circularized nucleic acid molecules based on interaction between target nucleic acid molecules and supplied oligonucleotides or fragments.

In another embodiment, the invention provides multiplex methods for detection and amplification of target polynucleotide sequences including mutation detection with circularized oligonucleotide molecules. In one aspect, the target nucleic acid molecule is circularized without prior amplification by PCR. Circularization of the target nucleic acid molecule may include circularization of the target, the complement thereof, or both. Free 3′ ends may be generated or supplied if needed or desired. The circularized nucleic acid molecule is then amplified by rolling circle amplification.

Multiple embodiments of the invention employ different methods of circularizing the target nucleic acid molecules (or complements thereof). Generally, the target nucleic acid molecules may be circularized by a number of different methods such as ligation using enzymes (such as T4 DNA ligase) or chemical methods, photochemical reactions, site specific or homologous recombination with enzymes (such as Cre-recombinase), and polymerase extensions in various forms. Circularization, such as by recombination using an enzyme such as Cre-recombinase, may require attachment of specific sequences to one or both ends of the target nucleic acid molecule (or complement thereof). In addition, the circularization methods of the invention may or may not require addition of specific sequences to one or both ends of the target nucleic acid molecule in a complex mixture (or complement thereof). The specific sequences being added to the ends of a target nucleic acid molecule are called the first linker nucleic acid molecule and the second linker nucleic acid molecule respectively. In one embodiment, a first linker nucleic acid molecule is affixed to the target nucleic acid molecule. The first linker nucleic acid comprises a sequence or moiety that allows it to be affixed to the target nucleic acid molecule. The first linker nucleic acid molecule may optionally comprise additional defined sequences that may by used later on in circularization, cloning, detection, amplification, or generation of RNA. Without limiting the generality of the foregoing, such defined sequences include restriction endonuclease sites, Cre-lox cross-over sites, RNA polymerase promoter sites, polymerase termination sites, hairpin loop structures, etc. For instance, the first linker may comprise a restriction endonuclease site, whereby sticky ends can be created at one or both ends of the target. The resulting target can be circularized if the two sticky ends are complementary, or by ligation with sticky ends of supplied hairpin loop primers.

In yet another embodiment, first linker nucleic acid molecule may be affixed by hybridization to the target nucleic acid molecule or by ligation to the target nucleic acid molecule. In embodiments using hybridization, the first linker nucleic acid molecule will have a complementary region on its 3′ end for hybridization. The complementary region may be, for example, a poly-T stretch that hybridizes to the poly-A tail of mRNA. Another example is a determined sequence if the sequence of the target nucleic acid is known. If the sequence of the target nucleic acid is unknown, then the complementary region may be randomized sequences of short length such as a hexamer, a heptamer, an octamer, a nonamer, a decamer, an undecamer, or a dodecamer to allow random hybridization. In certain embodiments, the first linker nucleic acid may be extended after hybridization to the target nucleic acid molecule by addition of a polymerase such as a reverse transcriptase if the target nucleic acid is RNA, and the first linker nucleic acid may comprise a hairpin structure for target circularization. In still another embodiment, the polymerase will add specific nucleotides to the end of a nascent strand once the polymerase has reached the end of the template stand. For example, MMLV reverse transcriptase will add cytosine nucleotides to the end of the nascent strand. Such overhangs may be used directly or for extension such as oligo switch to circularize the target nucleic acid to ensure that the full-length target nucleic acid is amplified. For instance, a hairpin loop oligo switch primer can be ligated to added cytosine nucleotides directly or as a template for further extension of the added cytosine nucleotides, and then ligated with the hairpin loop oligo switch primer. In other embodiments, terminal transferases are used to create such overhangs. In yet other embodiments, the first linker nucleic acid molecule may comprise a pool of linkers with a random sequence at the 3′ end and optional pre-selected arbitrary sequence, including defined structure sequence, at the 5′ end. Such first linker nucleic acid molecules may be used with single or double stranded target nucleic acid molecules. For single strand target nucleic acid molecules, the first linker nucleic acid molecules may have hairpin structure and be ligated to the both ends and then extended and ligated for circularization. For double strand target nucleic acid molecules, the random sequences will hybridize at the ends of the double stranded target nucleic acid molecule due to random unzipping of the ends of the double stranded target nucleic acid molecule as the nucleic acid “breathes”, or by denaturning the double strand. The first linker nucleic acid molecules performs as template for extending both ends of the double stranded target nucleic acid molecule to create overhangs for circularization. In addition, the first linker nucleic acid molecules may have hairpin structures and can be ligated to the ends of the double strand target nucleic acid molecules before or after or without extension for circularization. Thus, such linker nucleic acid molecules may be used to circularize the entire target nucleic acid molecules of unknown or known sequence.

In some embodiments, a second linker nucleic acid molecule is affixed to the target nucleic acid or complementary strand of target nucleic acid prior to or as a part of circularization of the target nucleic acid (or complement thereof). The second linker nucleic acid molecule comprises a sequence or moiety that allows it to be affixed to the target nucleic acid molecule. The second linker nucleic acid may optionally further comprise a region complementary to the first nucleic acid molecule to enable circularization by recombination such as by the Cre-LoxP system. For instance, LoxP sequences can be added to the both ends of the target mRNA by using polyT with LoxP sequence as RT primers and oligo switch primers with LoxP sequences. The resulting RNA DNA duplex with LoxP sequences at both ends can be circularized with Cre-recombinase. RNA can be nicked with Rnase H as primers for rolling circle amplification. In other embodiments, the second linker nucleic acid molecules may comprise hairpin structures with sticky ends and can be ligated with sticky ends of the target nucleic acid molecules to form a circular structure. In another embodiment, the second linker nucleic acid molecule may have a region that can hybridize to the first linker nucleic acid molecule to allow circularization of the target nucleic acid molecule by hybridizing the first linker nucleic acid molecule to the second linker nucleic acid molecule. As with the first linker nucleic acid molecule, the second linker nucleic acid molecule may further comprise additional regions and defined loop structure in assisting to circularize target nucleic acid molecules that have useful sequences such as restriction endonuclease sites, polymerase promoter sites, hairpin loop structures, etc. In yet another embodiment, the second linker nucleic acid molecule is added by the oligo switch method when the target nucleic acid molecule is mRNA. In such case, the oligo switch primer can be hairpins and covalently attached to the first strand cDNA by ligation or ligation after extension. This has the advantage of amplifying only full-length mRNA transcripts. For certain embodiments, a second linker nucleic acid molecule with a randomized sequence at its 3′ end can be added to the other end of the target nucleic acid by random hybridization of the second linker nucleic acid to the target nucleic acid molecule, followed by extension with polymerase.

In one aspect wherein the target nucleic acid molecule is mRNA the circularized nucleic acid molecule ideally includes full-length cDNAs. The circularized full-length cDNA may be amplified with supplied primers or randomers as primers to generate multiple copies of full-length double strand cDNA. The supplied randomers may have T7 promoter sequences at their 5′ ends. After exponential amplification, the resulting double strand products may be further amplified with T7 polymerase to generate RNA. In another aspects, the circularized full-length cDNA may be amplified with chimeric primers, for instance, having ribonucleotide in the 5′ end. Once the primers have hybridized and extended with the circular cDNA as template, Rnase H will nick the ribonucleotide sequence at the 5′ end. Then another primer hybridizes to replace the previous one for polymerization and extension, and the process will be cycled. In some embodiments, the circularized nucleic acid molecule will include an RNA polymerase promoter sequence such as the T7 RNA polymerase promoter. Depending upon the orientation and position of the T7 promoter, the amplified DNA can be used as a template to generate multiple copies of antisense RNA (aRNA) or of mRNA. By combining RNA transcription with rolling circle amplification, the aRNA or mRNA amplification efficiency is greatly enhanced compared to use of T7 polymerase in the absence of amplification. Furthermore, the methods of the invention can eliminate the 3′ bias and simplify the RNA amplification process. In still other embodiments, RNA polymerase promoters may be provided at both ends of the target nucleic acid molecule, incorporated into the circularized nucleic acid molecule in order to generate double stranded RNA. The resulting double strand RNA can be fragmented by Dicer for RNAi applications.

The invention also provides methods of amplification and detection of desired region of target nucleic acid molecules using rolling circle amplification. The first linker nucleic acid molecules or the second linker nucleic acid molecules or combination of both will be used to define the region of the target nucleic acid molecules to be amplified by hybridization or hybridization and ligation with the target in the desired region. Both first and second linkers comprise hairpin structures. The hairpin structures could be formed before or after they have hybridized with the target. The first and second linkers can be circularized with ligation if the target is present or after they have interacted with the target. Additional reaction steps such as polymerization may be necessary before ligation to form a circle. Furthermore the first and second linkers can be circularized in association with mutation detection based on whether the mutation in the target is present or not.

The target nucleic acid molecule may be circularized by a number of methods including, without limitation, blunt end ligation, annealing complementary ends followed by ligation, recombination between complementary regions, or annealing a primer with polymerase extension. The circularization will result in at least one strand of the nucleic acid being circularized. Circularization of an mRNA target nucleic acid molecule may be performed by self-priming after the reverse transcriptase to synthesize the second strand of the cDNA followed by closing the circle by self-ligation. A hairpin loop structure at the 5′ end of the first strand cDNA will further assist the self-ligation reaction. The product of such self-primed synthesis of the second strand is a double stranded cDNA molecule closed at the terminus corresponding to the 5′ terminus of the mRNA by a hairpin loop. In the same manner, self-priming can also be used to circularize single strand DNA.

The invention encompasses multiple methods of rolling circle amplification after the target nucleic acid molecule has been circularized. In some embodiments, a polymerase that can initiate at an appropriate promoter sequence is used. The promoter sequence may have been added in the first linker nucleic acid, the second linker nucleic acid, or the combination of the two. In certain embodiments, the polymerase needs a free 3′ end to begin polymerization. Such free 3′ end may be generated by a number of methods. In one embodiment, the 3′ end results from the circularization. In another embodiment, the 3′ end is generated after circularization by addition of one or more primers that hybridize to some portion of the circularized nucleic acid. In certain embodiments, the primers may be RNA:DNA chimeras. In some embodiments, randomers can be used as primers. In still other embodiments, the free 3′ end is introduced by nicking the circularized DNA randomly with limited amounts of endonucleases. In the case of amplification of a RNA, the RNA may be nicked with limiting amounts of RNaseH or the RNA may be completely removed with excess RNaseH. In still other embodiments, the free 3′ end is introduced by cutting with a restriction endonuclease at a hemi-methylated restriction site.

With a free 3′ end, the target nucleic acid may be amplified by rolling circle amplification in such embodiments needing a free 3′ end. Some embodiments include generation of an RNA transcript by adding an RNA polymerase that initiates transcription from a promoter added to the target nucleic acid molecule. In some embodiments, a single initiation point is used which results in linear amplification. This may be achieved through addition of a single primer, use of a single polymerase start point, or other generation of free 3′ ends on only one strand of the circularized nucleic acid molecule. In other embodiments, exponential amplification will be achieved by generation of free 3′ ends corresponding to both strands. An example would be to add a pair of primers each of which anneals to different strands of the circularized nucleic acid molecule.

In yet another embodiment, the circularized full-length targeted polynucleotide sequences can be constructed to contain regulatory elements to effect transcription and translation so that they can be used to express proteins in vivo or vitro, and/or signature sequences for specific applications such as detection tags. Such methods eliminate the complexity of inserting double strand cDNA into a vector or plasmid.

In yet another aspect of the invention, a target nucleic acid molecule is detected and/or amplified by addition of a circular nucleic acid molecule that comprises a first region that will hybridize to the target nucleic acid molecule. The target nucleic acid molecule is hybridized to the circular nucleic acid molecule, and rolling circle amplification is initiated at an extendable free 3′ end of the target nucleic acid molecule. The extendable free 3′ end may be generated in the target nucleic acid molecule before or after the target nucleic acid molecule has been hybridized to the circular nucleic acid molecule. This is particularly important when the target nucleic acid molecule is mRNA which has a poly-A tail at the 3′ end. The extendable free 3′ end may be generated by cleaving the target nucleic acid molecule prior to hybridization or after hybridization by site specific cleavage or by random nicking of the target nucleic acid molecule. One of skill in the art is aware of many methods of site specific cleavage, which are included in the invention. Examples include restriction endonucleases and, in the case of RNA, ribozymes, RNAi Dicer, etc. Random nicking may be performed with chemical agents or non-specific nucleases.

In one embodiment of the invention, the circular nucleic acid molecules can be constructed by using synthetic oligonucleotide with self-ligation, instead of template dependent ligation or by using a padlock probe. Such a method is a single molecular reaction or intramolecular reaction, which is more efficient and accurate compared to the use of padlock probes or template dependent ligation reactions.

In another embodiment of the invention, the full-length circular nucleic acid molecules of any gene can be constructed by using an existing full-length cDNA clone library with PCR amplification, or from RNA. The resulting full-length circular nucleic acid molecules can be used to amplify, detect and quantify specific genes or targets.

In yet another embodiment, the target nucleic acid molecules are detected and amplified by using circular nucleic acid molecular probes. The circular nucleic acid molecular probes may hybridize with the target, and may contain target sequence. The free 3′ ends can be selectively generated from supplied DNA fragments, RNA fragments, or RNA DNA chimeric fragments. Additional reaction steps after interaction between target and added fragments, such as Rnase H nicking, polymerase reaction or other reactions may be used to generate free 3′ end. The free 3′ ends are only generated for polymerization and detection with circular nucleic acid molecules as template only when the target nucleic acid is present or when mutation in the target nucleic acid molecules are present or not. The added fragments can be linear, hairpin or circular with or without defined structures. There might be more than one fragments interact with target nucleic acid molecules simultaneously. The process of the interaction between added fragments and target nucleic acid molecules may be cycled to repeatedly generate free 3′ end for rolling circle amplification. The target nucleic acid molecules can be single stranded DNA, double stranded DNA or RNA. Examples can be found in FIG. 5D, 5E, 5F. Hence, the method may be used to selectively amplify targets which have a particular sequence, such as a mutation or lack thereof, wherein RCA will only be initiated on circularized nucleic acid molecules with the specific sequence.

Once rolling circle amplification has been initiated at a free 3′ end, additional primers complimentary to the nascent strand may be used to further enhance amplification.

In yet another aspect of the invention, mutations such as single nucleotide polymorphisms in the target nucleic acid molecules can be detected by selectively generating free 3′ ends available for RCA only in the mutant or non-mutant target nucleic acid molecule. Examples include ribozymes targeted at the site of the mutation, and hybridization of the target nucleic acid molecule with nucleic acid molecules complementary to the target nucleic acid molecule with or without the mutation followed by nicking with enzymes such as S1 nuclease that will cleave at mismatches.

In yet another aspect of the invention, the target molecules can be single strand or double strand DNA. A fragment of RNA can be added where the 3′ extension has been blocked. If the targeted nucleic acid molecules is present, the RNA fragment will hybridize to the target and then any enzymes such as RNaseH will digest the added fragment of RNA to generate the free 3′ end. Thereafter the added circular nucleic acid molecules will initiate the rolling circle amplification. The RNA fragment may contain a hairpin loop to increase the reaction specificity.

Detection of the amplified product may be performed by any method applicable to the detection of nucleic acids, such as those described below.

Many embodiments of the invention may be practice on a solid phase substrate. Suitable solid-phase substrates include any solid material to which sequences (targets, probes, supplied fragments, etc.) can be coupled or adhered, including materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Suitable solid substrates can have any useful form including thin films or membranes, beads, bottles, dishes, slides, fibers, woven fibers, shaped polymers, particles and microparticles. Preferred forms for a solid substrates are microtiter dishes and glass slides, particulary a microarray slide to which up to 256 separate target samples have been adhered as an array of small dots. Each dot is preferably from 0.1 to 2.5 mm in diameter, and most preferably around 2.5 mm in diameter. Such microarrays can be fabricated using well-known methods of photolithography, contact deposition and ink jet printing, etc.

Sequences immobilized on a solid substrate allow formation of target-specific amplified nucleic acid localized on the solid-state substrate. Such localization provides a convenient means of washing away reaction components that might interfere with subsequent detection steps, and a convenient way of assaying multiple different samples simultaneously. Amplified nucleic acid can be independently formed at each site where a different sample is adhered. The disclosed method can be used for immobilization of target sequences or other oligonucleotide molecules to form a solid-state sample.

### BRIEF DESCRIPTION OF THE FIGURES

FIG. 1 summarizes several invention embodiments for amplifying RNA using RCA.

FIGS. 2 and 3 summarize invention embodiments for circularizing RNA and DNA templates.

FIG. 4 summarizes how to use RCA for SNP detection and how to amplify a specific gene segment.

FIG. 4 summarizes methods to amplify DNA with RCA.

FIG. 5 summarizes detection RNA and DNA with circular probes.

### DETAILED DESCRIPTION OF THE INVENTION

As used herein, a “circular nucleic acid molecule” is a nucleic acid molecule with at least one contiguous strand. The circular nucleic acid molecule is used to detect and amplify a target nucleic acid molecule. At least a portion of the target nucleic acid molecule may be contained within or complementary to a portion of the circular nucleic acid molecule. The circular nucleic acid molecule may be RNA, DNA, PNA, or any combination thereof. The circular nucleic acid molecule may contain any natural or unnatural bases and may have missing bases. The circular nucleic acid molecule may be generated by any suitable techniques, including without limitation, synthetic and natural methods.

As used herein, a “circularized nucleic acid molecule” is a nucleic acid molecule that contains or is complementary to the target nucleic acid sequence within the circular portion. The circularized nucleic acid molecule is generated as a part of the amplification and cloning process. The circularized nucleic acid molecule comprises at least one contiguous strand. The circularized nucleic acid molecule may be RNA, DNA, PNA, or any combination thereof. The circularized nucleic acid molecule may contain any natural or unnatural bases and may have missing bases.

As used herein, a “free 3′ end” is a 3′ end of a nucleic acid molecule that is annealed to a template nucleic acid strand that a polymerase may extend.

As used herein, a “target nucleic acid molecule” is the nucleic acid molecule to be cloned, amplified or detected through rolling circle amplification. The target nucleic acid molecule may be obtained from any source. It can be mRNA, rRNA, RNAi, RNA being processed, genomic DNA, cDNA, etc.

Preparation of the Target Nucleic Acid

The invention includes target nucleic acid molecules that are to be amplified for cloning, detection, etc. The disclosed methods may be adapted to any nucleic acid molecule of interest. The nucleic acid may be obtained from any source including, without limitation, cellular or tissue samples, nucleic acid molecules in libraries, chemically synthesized nucleic acid molecules, genomic nucleic acid molecules, cloned nucleic acids, mixtures of such nucleic acids, messenger RNAs, including splice variants and intermediates. The methods are particularly suited to generating libraries from mixtures of mRNAs. The only requirement is that the nucleic acid be amenable to circularization according to the methods of the invention or have a defined sequence for detection by the methods of the invention.

The nucleic acids may be obtained by a wide range of methods available to one of skill in the art. Detailed protocols for numerous such procedures are described in, e.g., in Ausubel et al. Current Protocols in Molecular Biology (Supplemented through 2000) John Wiley & Sons, New York; Sambrook et al. Molecular Cloning—A Laboratory Manual (2nd Ed.), Vol. 1-3, Cold Spring Harbor Laboratory, Cold Spring Harbor, N.Y., 1989, and Berger and Kimmel Guide to Molecular Cloning Techniques, Methods in Enzymology volume 152 Academic Press, Inc., San Diego, Calif.

Once the nucleic acid molecules of interest have been obtained, the molecules may be further manipulated depending upon the later methods applied to the molecules. For example, when detecting a target nucleic acid molecule with a circular nucleic acid, the target nucleic acid may be pre-treated to generate different or additional free 3′ ends. Examples include targeted cleavage with a site-specific ribozyme or hybridization to a complementary nucleic acid sequence and digestion with the appropriate nuclease. In the case of mRNA, the poly-A tail may be removed by any suitable technique known to one of ordinary skill in the art. An example for mRNA would be to add single stranded polyT DNA and RNAseH. In addition, longer nucleic acid molecules may be cleaved to generate shorter fragments. Examples of such cleavage include physical shearing of the DNA by pipetting or sonication, digestion with restriction endonucleases, etc.

In addition, to assist in circularization, the nucleic acid may be treated with a variety of other protocols such as filling in over-hangs generated by restriction enzymes, adding phosphate groups with poly-nucleotide kinases for later ligation or removing phosphate groups with phosphatases to prevent later ligation. The cohesive ends generated by restriction endonucleases may be annealed and ligated to circularaize the nucleic acid for rolling circle amplification. As discussed above, one of skill in the art may find detailed protocols for all such procedures in the literature.

Circularization of the Target Nucleic Acid

The invention includes circularization by ligation, hybridization and ligation, hybridization and polymerization and ligation, recombination, and chemical reaction, and photoreaction. Select an appropriate ligase for the particular reaction is routine in the art: DNA ligases for DNA nucleic acids, RNA ligases for RNA, etc. Suitable ligases include T4 RNA ligase to circularize single strand DNA or RNA and T4 DNA ligase (Davis et al., Advanced Bacterial Genetics—A Manual for Genetic Engineering (Cold Spring Harbor Laboratory, Cold Spring Harbor, N.Y., 1980)), E. coli DNA ligase (Panasnko et al., J. Biol. Chem. 253:4590-4592 (1978)), AMPLIGASE.RTM. (Kalin et al., Mutat Res., 283(2):119-123 (1992); Winn-Deen et al., Mol Cell Probes (England) 7(3):179-186 (1993)), Taq DNA ligase (Barany, Proc. Natl. Acad. Sci. USA 88:189-193 (1991), Thermus thermophilus DNA ligase (Abbott Laboratories), Thermus scotoductus DNA ligase and Rhodothermus marinus DNA ligase (Thorbjarnardottir et al., Gene 151:177-180 (1995)). T4 DNA ligase is preferred for ligations involving RNA target nucleic acid molecules due to its ability to ligate DNA ends involved in DNA:RNA hybrids (Hsuih et al., Quantitative detection of HCV RNA using novel ligation-dependent polymerase chain reaction, American Association for the Study of Liver Diseases (Chicago, Ill., Nov. 3-7, 1995)).

Circularization of the target nucleic acid molecule may include circularizing the target (including a desired region or subset of the target molecule), or the complement thereof, or the complementary strand of the complementary strand of the target, or a combination of the target and the complement thereof, or a combination of the complementary strand of the target and the complementary of the complementary strand of the target.

Attachment of specific sequences to one or both ends of the target or complementary strand of the target or combination of the target and complementary strand of the target may be used to circularize the target. The specific sequences added to the ends of a target are called the first and second linker nucleic acid molecules. After the first linker or the second linker has been affixed to the target, additional reactions such as hybridization, ligation, polymerization and ligation or restriction enzyme reaction and ligation may be used to circularize the target.

Affixing linkers to the target includes attaching arbitrary sequences with defined structures or attaching reactive functional groups to the desired region of the target or complementary strand of target so that the entire or portion of the target or the complementary strand of the target can be circularized and amplified by RCA.

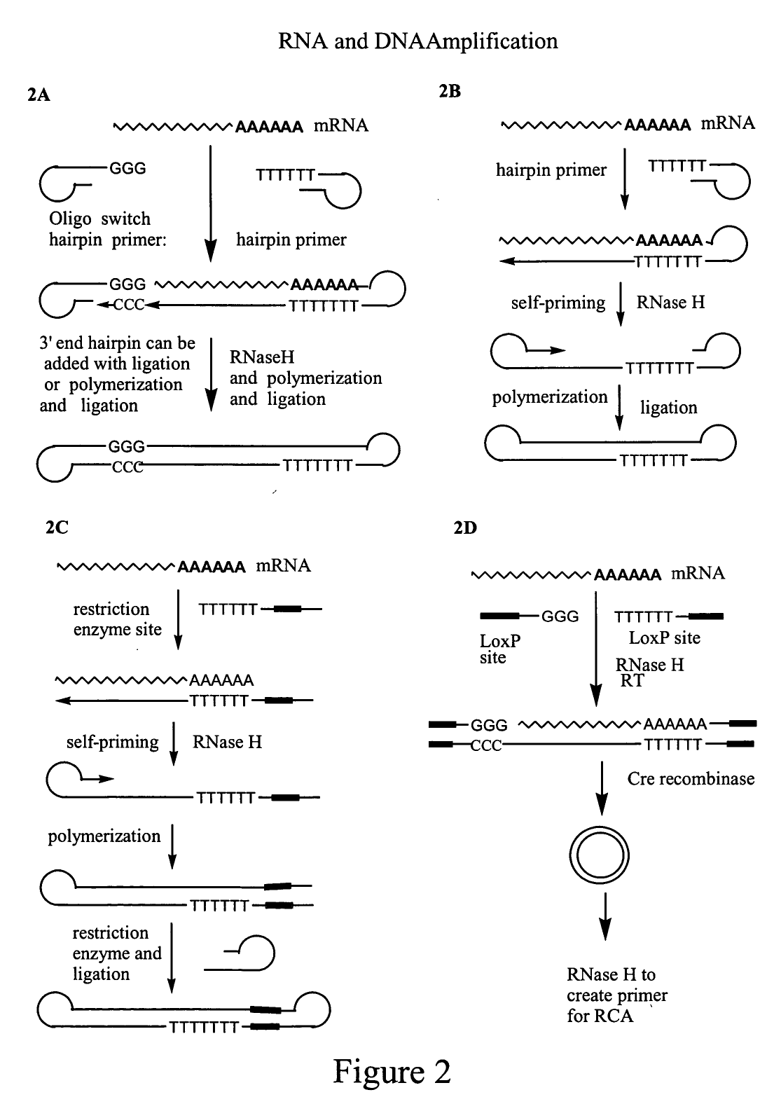

The first linker nucleic acid molecule may be affixed to the target nucleic acid molecule by a range of techniques known to those of ordinary skill in the art. It is preferred that the first linker nucleic acid molecule be affixed to an end of the target nucleic acid. In certain embodiments, a first linker nucleic acid molecule may be affixed at both ends of the target nucleic acid. Methods of affixing the first linker to the target include ligation, hybridization and ligation, and hybridization followed polymerase extension and other enzymatic reactions such as terminal transferase, and ligase. A preferred example is a first linker comprising a poly-T sequence at its 3′ end for mRNA targets. In addition, the linker may comprise one or more of a number of other sequences, optionally with predetermined, defined structures that facilitate circularization, detection, etc. Examples include RNA and/or DNA polymerase promoters, site specific recombination sequences such as loxP, homologous sequences for general recombination, restriction sites (including hemimethylated sites), transcription termination sites, ribosome binding sites, ribozymes, RNAi, replication origins, genes, including ORFs, hairpin structures, etc. Optionally, a second linker may be affixed by methods similar to those above. The second linker may comprise one or more of a number of other sequences that may be useful for circularization, detection, etc. In addition, when the target nucleic acid molecule is mRNA, the second linker may be affixed by the CAP-switch method (e.g. U.S. Pat. No. 5,962,271). In addition, the second linker may be affixed by the oligo-capping method (e.g. U.S. Pat. No. 5,597,713). The second linker may also be affixed by hybridization in a manner that facilitates template switching, such as described by Patel et al. PNAS, 93:2969-2974. The CAP switch template or template switching oligo nucleotides may have hairpin structure and be ligated to the target, or may have restriction enzyme sites to generate sticky ends for circularization. Examples may be found in FIG. 2A.

In specific embodiments, the first and/or second linker have hairpin structure. With hairpin structure in the first linker or second linker, the target can be circularized by creating a second hairpin in a desired region of the target and then reacting with first hairpin to form a circle. Reactions for circularizing the target between the first and second hairpins include steps of hybridization, polymerization or ligation or combination of hybridization, polymerization and ligation. The second hairpin may be generated by ligation or hybridization or combination of both hybridization and ligation or self-priming. Examples may be found in FIG. 2A, 2B.

In specific embodiments, the first and/or second linker contain one or more restriction enzyme sites. With a restriction enzyme site in the first and/or second linker, sticky ends can be generated in one or both ends of the target. The target can be circularized by ligation with one or two sticky ends of hairpin fragments. Alternatively, the two sticky ends of the target are complementary and ligated to form a circle. Example may be found in FIG. 2C, 3B.

In a specific embodiment, the first and/or second linker contain homologous sequences. The target with homologous sequences at both ends can be circularized with recombinase. Examples may be found in FIG. 2D.

For single or double stranded nucleic acid molecules, a first and optionally a second linker nucleic acid molecule may comprise random sequences if the target sequence is unknown, or defined sequences if the target sequence is known at their 3′ ends which can hybridize and ligated to the target nucleic acid molecule or hybridize and be extended and ligated by addition of an appropriate polymerase. The linker nucleic acid molecules may comprise additional sequences with defined structures such as hairpins for circularization. The first linker or optionally the second linker with hairpin structures will hybridize and be ligated at the ends of double stranded nucleic acid molecule due to random unzippingof the double helix at the ends from breathing of the duplex or denaturation of the duplex Once the strand has opened and the linker has been hybridized or ligated, a polymerase may extend from the free 3′ end. Ligating 3′ end and 5′ end will form a circle. Alternatively, the first linker or optionally the second linker with restriction enzyme sites will hybridize at the ends of double stranded nucleic acid molecule due to random unzippingof the double helix at the ends from breathing of the duplex. Once the strand has opened and the linker has hybridized, a polymerase may extend from the free 3′ end of the target nucleic acid molecules to generate blunt ends. The resulting double strand targeted nucleic acid molecules can be then circularized and amplified. Examples may be found in FIG. 3C, 3D.

The target nucleic acid molecule may be circularized by hybridization. The affixed first linker may hybridize to the other end of the target nucleic acid molecule or to an optionally affixed second linker. One of skill in the art will recognize that the invention may optionally include additional intervening linkers as desired. Once hybridized, ligase should be used to generate at least one strand that has been ligated into a contiguous molecule. The target nucleic acid molecule may also be circularized by chemical reaction or photo-reaction. Examples may be found in FIG. 3A.

Additionally, mRNA target nucleic acid molecules may be circularized by self-priming of the reverse transcription reaction to generate the second DNA strand. Once the strand has been synthesized, the circle may be closed by ligation. In some embodiments, the first linker nucleic acid will have a hairpin to enhance circularization after self-priming. The hairpin loop may be of arbitrary size and can accommodate any additional sequence elements that may be desirable. In some embodiments, the first linker nucleic acid will have a restriction enzyme site. The sticky ends can be generated after the second strand DNA synthesis. Then the circle may be closed by ligation with a sticky end of hairpin fragment. Examples may be found in FIG. 2B, 2C.

Additionally, the first strand cDNA synthesis from mRNA may be not full-length. In such case, a first and optionally a second linker nucleic acid molecule may comprise random sequences at their 3′ ends which can hybridize to the 3′ end first strand cDNA and be extended by addition of an appropriate polymerase. The first strand cDNA can be synthesized with modified polyT or modified random primers with or without hairpin structures or with or without restriction enzyme sites. The linker nucleic acid molecules may comprise additional sequences for circularization.

With hairpin structure, the random sequences can hybridize at the 3′ end of first strand cDNA due to random unzipping of the RNA: DNA duplex at the 3′ end of the first strand from breathing of the duplex or denaturation of the duplex. Once the strand has opened and the linker has been hybridized or ligated, a polymerase may extend from the free 3′ end. Ligating the 3′ and 5′ ends will form a circle. Alternatively, the first linker or optionally the second linker containing restriction enzyme sites will hybridize at the ends of RNA DNA duplex due to random unzipping of the duplex at the ends from breathing of the duplex. Once the strand has opened and the linker has hybridized, a polymerase may extend from the free 3′ end of the first strand cDNA to generate blunt ends. The resulting double stranded target can be then circularized and amplified. Examples may be found, for example, in FIG. 14 of our priority application.

In addition, the target nucleic acid may be circularized by recombination. Such recombination may be accomplished with a site-specific recombinase such as Cre or through a recombinase that will recombine molecules with homologous sections (e.g. recombination with LoxP-CreI, U.S. Pat. No. 5,591,609). Examples may be found in FIG. 2D.

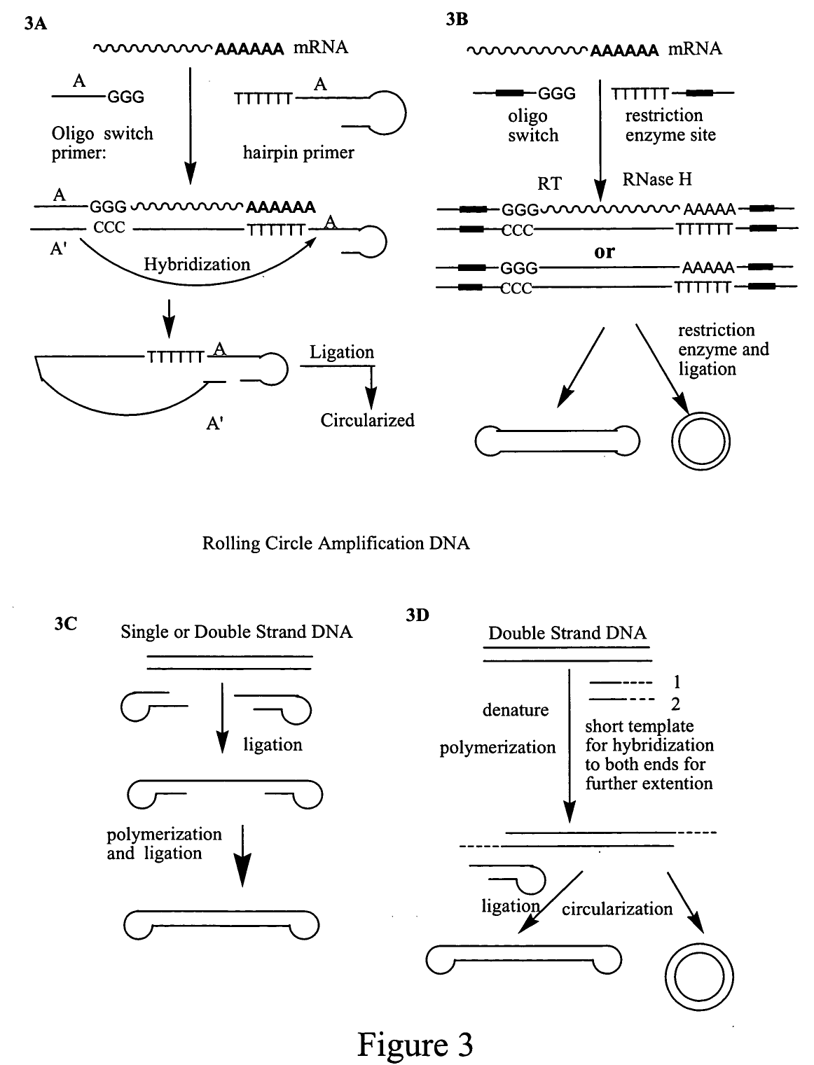

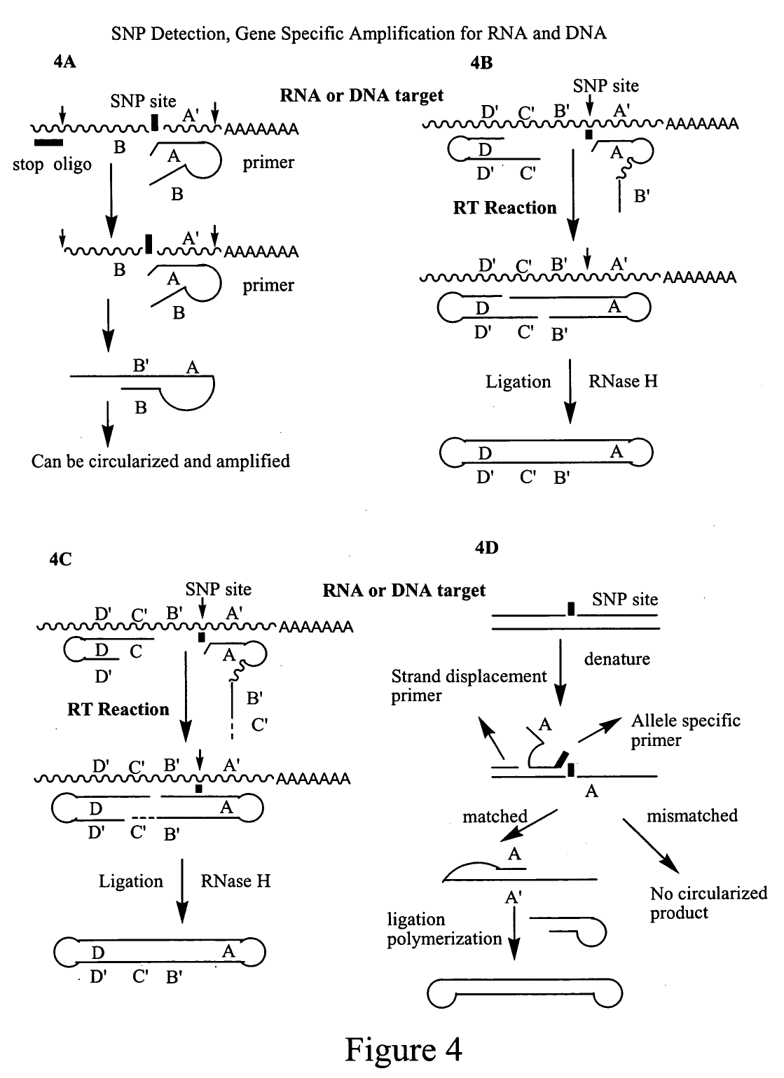

In one embodiment, to amplify and detect a desired region of a target using RCA, the first and/or second linker may be used to define the region of the target to be amplified by hybridization or hybridization and ligation with the target in the desired region. The first and/or second linkers comprise hairpin structures, which may be formed before or after they have hybridized with the target. The first and second linkers can be circularized with ligation if the target is present or after they have interacted with the target. Additional reaction steps such as polymerization may be used after hybridization before ligation to form a circle. In addition the first and second linkers can be circularized in association with mutation detection based on whether the mutation in the target is present or not. Examples may be found in FIGS. 4A, 4B and 4C, 4D.

The first and/or second linkers may hybridize with the target to form a triple helix and be ligated to form a circle. The RCA may release the target to be available for second round hybridization and circularization and amplification.

The ligated circular nucleic acid molecules can be cut to from linear products, which can be amplified with PCR instead of rolling circle amplification.

Generation of free 3′ ends.

Once the target has been circularized, a free 3′ end is needed begin RCA, which may need to be generated from the target or supplied. The free 3′ end be generated from target before or after the target has been circularized. For methods that involve circularization of the target nucleic acid, the circularization itself may result in free 3′ ends. Many methods are available for generating free 3′ ends if needed. Where the target nucleic acid is an RNA molecule, and it is hybridized to a complementary DNA molecule, RNAseH may be used to digest the RNA molecule entirely, or limiting amounts of RNAseH may be used to nick the RNA and generate free 3′ ends. Furthermore, limiting amounts of any endonuclease may be used to nick double stranded nucleic acid molecules. The limiting amount is controlled such that both strands are not nicked because at least one strand must remain an intact circle for rolling circle amplification. Similarly, limiting amounts of chemicals that nick DNA may be used to generate free 3′ ends.

In addition, specific free 3′ ends may be generated by use of thiophosphorylated or hemimethylated double stranded nucleic acids. Certain restriction endonucleases will cut only one strand when the restriction site is thiophosphorylated or hemimethylated. Such hemimethylated DNA may be generated by a number of methods. For example, nucleic acid molecules may be chemically synthesized with methylated nucleotides at key positions; the nucleic acid molecule may be methylated with site specific DNA methylases in vitro; or the nucleic acid molecule may be obtained from an organism that expresses the requisite site-specific DNA methylase. Also, certain restriction endonucleases may be used that naturally only cut one strand of a duplex, e.g., N.Alw I, N.BstNB I (both available from New England Biolabs).

Additionally, ribozymes or RNAi constructs such as Dicer may be used to cleave the ribonucleic acid molecules at specific locations, thus generating free 3′ ends.

Another method of generating free 3′ ends is by supplying an oligonucleotide primer. A preferred primer is a strand displacement primer. One form of strand displacement primer is an oligonucleotide having sequence complementary to a strand of a circular nucleic acid. This sequence is referred to as the matching portion of the strand displacement primer. The matching portion of a strand displacement primer may be complementary to any sequence. However, it is preferred that it not be complementary to any additional strand displacement primers, if such are being used. This prevents hybridization of the primers to each other. The matching portion of a strand displacement primer may be complementary to all or a portion of the inserted nucleic acid molecule, although this is not preferred. The matching portion of a strand displacement primer can be any length that supports specific and stable hybridization between the primer and its complement. Generally this is 12 to 35 nucleotides long, preferably 18 to 25 nucleotides long.

It is preferred that strand displacement primers also contain additional sequence at their 5′ end that does not match any part of a strand of the circular nucleic acid. This sequence is referred to as the non-matching portion of the strand displacement primer. The non-matching portion of the strand displacement primer, if present, serves to facilitate strand displacement during DNA replication. The non-matching portion of a strand displacement primer may be any length, but is generally 1 to 100 nucleotides long, and preferably 4 to 8 nucleotides long.

Optionally, the strand displacement primers may also contain additional RNA sequence at the 5′ end of that may or may not match any part of a strand of the circular nucleic acid. Examples of use of such chimeric primers are disclosed in U.S. Pat. No. 6,251,639 and U.S. Pat Appl 2003/0087251.

Additional strand displacement primers may be used to increase the amplification of the target nucleic acid. The additional strand displacement primers may be complementary to the same strand that the first strand displacement primer complements to linearly increase the amplification, or have the same sequence as the strand that the first strand displacement primer complements to geometrically increase the amplification. Again, it is preferred that no primer strand displacement primer is complementary to any other strand displacement primer to prevent the primers from hybridizing to one another.

Strand displacement primers may also include modified nucleotides to make them resistant to exonuclease digestion. For example, the primer can have three or four phosphorothioate linkages between nucleotides at the 5′ end of the primer. Such nuclease resistant primers allow selective degradation of excess unligated linear vectors that might otherwise interfere with hybridization of probes and primers to the amplified nucleic acid. Strand displacement primers can be used for strand displacement replication and strand displacement cascade amplification, both described below.

Additionally, the free 3′ ends may be provided by addition of short oligonucleotides of random sequence. The preferred length is hexamers. To assist strand displacement, the nucleotides at the 5′ end may be RNA.

Amplification by Rolling Circle Amplification

Rolling circle amplification may be performed with the circularized nucleic acid molecules and circular nucleic acid molecules of the invention. This reaction requires the two components: (a) a free 3′ end, and (b) a rolling circle polymerase. The polymerase catalyzes primer extension and strand displacement in a processive rolling circle polymerization reaction that proceeds as long as desired, generating a molecule of up to 100,000 nucleotides or larger. This reiterated DNA sequence DNA (R-DNA) consists of long repeats of the circular or circularized nucleic acid molecule sequence. A number of references disclose primer, primer design, and amplification techniques, including U.S. Pat. Nos. 5,871,921, 5,648,245, 5,866,377 and 5,854,033; see also, US Pat Appl. No. 20030165948.

Detection with a Circular Nucleic Acid Probes

The invention also includes detection of target nucleic acid molecules using circular nucleic acid molecules as probes to detect such target nucleic acid molecules based on whether RCA has happened or not. The probes may comprise entire or portions of target sequences, or complementary strands thereof based on the source of the free 3′ end to hybridize to the probe to initiate RCA. If the free 3′ end is generated from the target or the complement thereof to initiate the RCA, the probes comprise and/or hybridize with a portion of or entire target sequence or complement thereof. If the free 3′ end is not generated from the target or complement thereof, the probe does not have to comprise and/or hybridize with a portion of or or entire target sequence or complement thereof.

The first part of the probe contains a sequence that will hybridize to the target nucleic acid molecule of interest, and the second part contains a sequence that enables detection of the amplified circular.

In one aspect of the invention, the probes can be constructed from linear short oligo fragments with self-ligation. The linear short oligo fragments may contain special hairpin structures so that they can be self-ligated to form circular nucleic acid molecules. Such a method offers advantages compared to padlock probes and additional template. The ligation efficiency is much higher, and it avoids mis-ligation to form larger linear strand or larger circular nucleic acid molecules.

In another aspect of the invention, a full-length circular cDNA nucleic acid molecule can be constructed from a commercially available full-length cDNA clone library. A gene specific clone is selectively amplified with PCR and then circularized by self-ligation. The resulting full-length circular cDNA nucleic acid molecules can be used for gene specific detection and amplification without needing to use TaqMan or RT-PCR.

In one embodiment, the probes may optionally comprise additional defined sequences that may by used in subsequent cloning, detection, amplification, or generation of RNA. Such defined sequences include restriction endonuclease sites, RNA polymerase promoter sites, polymerase termination sites, randomized sequences of short length such as a hexamer, a heptamer, an octamer, a nonamer, a decamer, an undecamer, or a dodecamer.

The invention encompasses compositions and methods useful for the amplification to RNA by reverse transcriptase-rolling circle amplification (RT-RCA) in a simplified procedure using combinations of reverse transcriptase and isothermal strand-displacement enzymes. The RNA is transcribed to generate cDNA with reverse transcriptase. The cDNA is circularized and amplified with isothermal strand-displacement enzymes. The amplified products can be transcribed to generate RNA if T7 promoter is present. Such RNA amplification methods combine the amplification efficiency from both rolling circle amplification and T7 amplification. The invention thus facilitates the rapid and efficient amplification of nucleic acid molecules and the detection and quantification of RNA molecules. The invention also is useful in the rapid production and amplification of cDNAs (single-stranded and double-stranded) which may be used for a variety of industrial, medical and forensic purposes.

The probe may also be constructed by circularizing the target nucleic acid molecules; applicable methods to circularize the target nucleic acid molecules are described herein.

Once the target nucleic acid molecule is hybridized to the circular nucleic acid, rolling circle amplification can be initiated using the target nucleic acid molecule as a free 3′ end to initiate rolling circle amplification. Suitable methods to generate free 3′ end from target to initiate the rolling circle amplification are described herein. In contrast prior methods of detection using rolling circle amplification rely upon ligation to form the circular nucleic acid. However, in certain embodiments, a linear strand may be used for detection that needs to be ligated for RCA to begin. This may be used to increase the sensitivity of detection. The invention utilizes a nucleic acid that has already been circularized prior to addition to the sample and the target nucleic acid itself provides the free 3′ end. The above methods of generating a free 3′ end may be used to generate one or more free 3′ ends as desired. One of skill in the art will recognize that primers may be used to provide free 3′ ends as long as care is taken in the design of such primers that the primer will not hybridize to the circular nucleic acid and allow RCA directly. Thus, the primer may have a sequence at its 3′ end that is the same as the circular nucleic acid. Such primers will allow (n!) factorial amplification. It is preferred that the primer not hybridize to the target nucleic acid molecule. In one embodiment, the circular nucleic acid molecule further comprises a poly-A portion. This embodiment may be used in detection of an mRNA of interest. Once the target nucleic acid of interest has bound to the circular nucleic acid and rolling circle amplification has begun, the poly-A tails of the mRNA will bind to the nascent nucleic acid with the complementary poly-T portion. Thus, in such embodiment, (n!) factorial amplification may be achieved without addition of any primers.

The methods of the current invention may also be applied to detection of mutations. The method of generating free 3′ ends and the reaction conditions may be selected whereby only mutant target nucleic acid molecules are amplified or only non-mutant target nucleic acid molecules are amplified. One example is targeted degradation of RNA by RNAseH using DNA hairpins. If the target nucleic acid molecule carries a single nucleotide polymorphism, the DNA at the hairpin will not anneal and RNAseH will not digest the RNA and generate a free 3′ end. Thus, RCA will not be initiated by mutant target nucleic acid molecules.

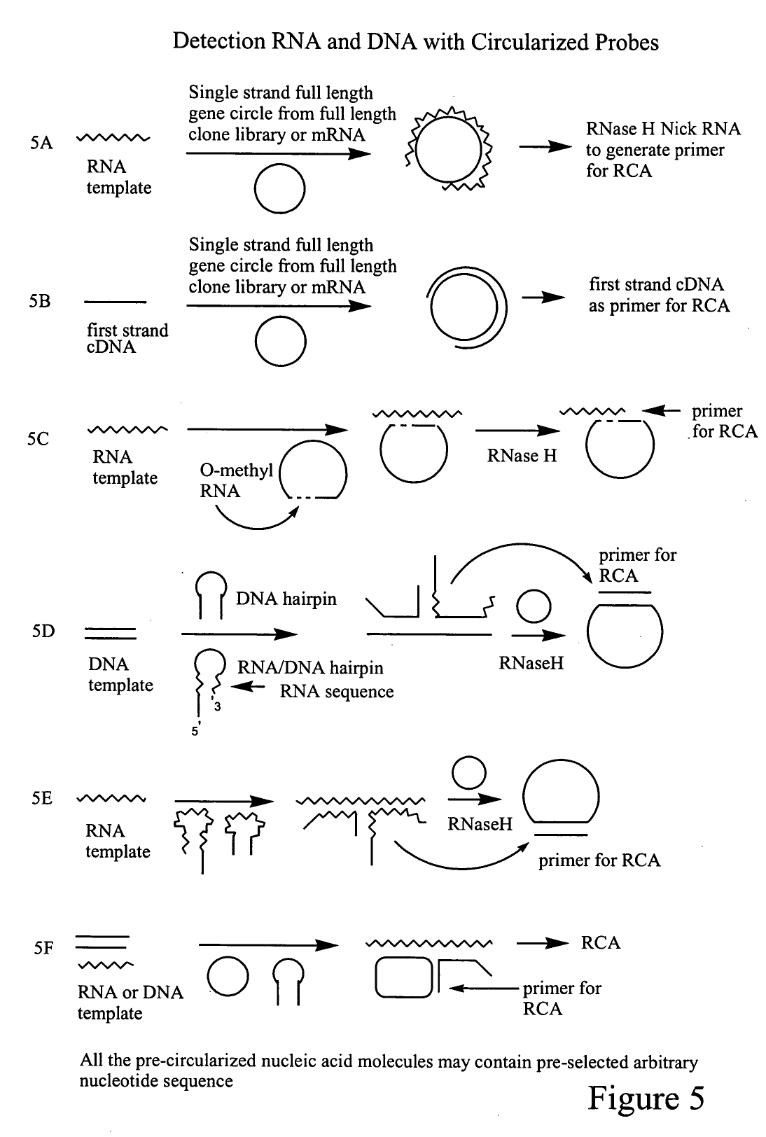

The invention provides alternative methods for detecting and/or amplifying target nucleic acid molecules by using circular nucleic acid molecular probes. The probes may hybridize with the target, and may contain target sequence. The free 3′ ends can be selectively generated from non-target sequences of supplied DNA fragments, RNA fragments, or RNA DNA chimeric fragments resulting from interaction between target nucleic acid molecules and the supplied fragments. Additional reaction steps after interaction between target and the supplied fragments, such as Rnase H nicking, polymerase reaction, transcription, restriction enzyme cut, or other reactions may be used to generate free 3′ end. Free 3′ ends are generated for polymerization and detection with circular nucleic acid molecules as template when the target nucleic acid is present or when a defined mutation in the target nucleic acid molecules is present or not. The supplied fragments can be linear, hairpin or circular with or without defined structures, and more than one fragment can be used to interact with the target simultaneously. The process of the interaction between supplied fragments and target nucleic acid molecules may be cycled to repeatedly generate free 3′ ends for rolling circle amplification. The target nucleic acid molecules can be single stranded DNA, double stranded DNA or RNA. Examples may be found in FIG. 5D, 5E, 5F. The probes may optionally comprise additional defined sequences that may be used in subsequent cloning, detection, amplification, or generation of RNA. Such defined sequences include restriction endonuclease sites, RNA polymerase promoter sites, polymerase termination sites, randomized sequences of short length such as a hexamer, a heptamer, an octamer, a nonamer, a decamer, an undecamer, or a dodecamer.

Detection of amplified product may be performed using any suitable technique for detection of nucleic acids. A few examples of detection methods are dyes that either directly or though an additional linked moiety interact with the nucleic acid by covalent linkage, intercalation, or some other form of binding. Radiolabels may also be used.

The detection methods of the invention may also be used in multiplexed reactions, i.e., the simultaneous detection of two or more nucleic acids in a single sample. One of skill in the art may employ any suitable method for multiplexed detection of nucleic acids. Typically, the products of the rolling circle amplification are differentiable, which may be due to incorporation of different labels into the amplified products, different lengths of the products, or different sequences of the products. An example of a method of incorporation of different labels is to use different secondary primers with label attached. In designing circular nucleic acids for multiplexed detection, it is preferred that the regions complementary to the target nucleic acids be substantially different to limit non-specific priming of the rolling circle amplification reaction. Ideally, any circular nucleic acid should be designed to limit non-specific priming by non-target nucleic acids that may be in a mixture with the target nucleic acid.

Detecting Products

To aid in detection and quantification of nucleic acids amplified using rolling circle amplification for cloning or detection of nucleic acids, detection labels can be directly incorporated into amplified nucleic acids or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid or antibody probes are known to those of skill in the art. Examples of detection labels suitable for use in rolling circle amplification are radioactive isotopes, fluorescent molecules, phosphorescent molecules, enzymes, antibodies, nucleic acid binding proteins, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 rim), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

Labeled nucleotides are preferred form of detection label since they can be directly incorporated into the products of rolling circle amplification during synthesis or affixed after synthesis. Examples of detection labels that can be incorporated into amplified nucleic acid products include nucleotide analogs such as BrdUrd (Hoy and Schimke, Mutation Research 290:217-230 (1993)), BrUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boehringher Mannheim). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified nucleic acids, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-(1,2,-dioxetane-3-2′-(5′-chloro)tricycle(3.3.-1.1.sup.3,7)decane)-4-yl)phenyl phosphate; Tropix, Inc.).

A preferred detection label for use in detection of amplified RNA is acridinium-ester-labeled DNA probe (GenProbe, Inc., as described by Arnold et al., Clinical Chemistry 35:1588-1594 (1989)). An acridinium-ester-labeled detection probe permits the detection of amplified RNA without washing because unhybridized probe can be destroyed with alkali (Arnold et al. (1989)).

Molecules that combine two or more of these detection labels are also considered detection labels. Any of the known detection labels can be used with the disclosed probes, tags, and method to label and detect nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. Such methods can be used directly in the disclosed method of amplification and detection. As used herein, detection molecules are molecules that interact with amplified nucleic acid and to which one or more detection labels are coupled.

Figure Descriptions

The invention encompasses methods depicted in the Figures, including methods for circularizing target mRNA for amplification, such as shown in FIG. 2, panels A, B and D, FIG. 3, panel B, and FIG. 4, panel B. These include methods for circularizing mRNA, preferably full-length mRNA, using self-priming and/or oligo switch template, and methods of amplifying one or more mRNA segments, mixtures or individual mRNA molecules. The invention also encompasses method which use pre-circularized nucleic acid molecules for mRNA and DNA detection and amplification, such as shown in FIG. 5, panels A, B and F. All the subject methods may be practiced in conjunction with disclosed methods of multiplex detection and amplification.

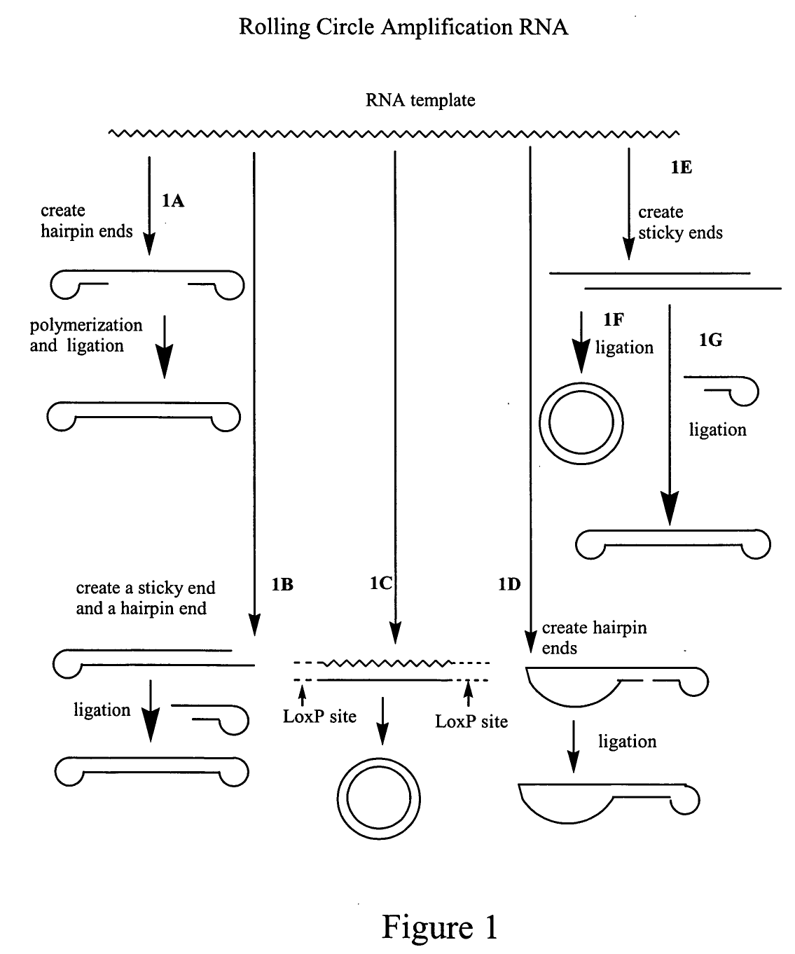

FIG. 1 summarizes invention embodiments for circularizing RNA templates. In panel 1A the RNA template is converted to single strand cDNA with hairpins at both the 3′ end and the 5′ end. To form a circle, the second strand cDNA is synthesized by polymerizing a 3′ end hairpin and subsequently ligating it with the 5′ end hairpin. The resulting circular cDNA is amplified by RCA. The hairpins at 3′ and 5′ ends may further comprise a functional sequence, such as a detection sequence, a site specific recombination sequence, a sequence for homologous recombination, a restriction endonuclease sequence, a promoter sequence, a transcription termination sequence, a ribosome binding sequence, a ribozyme sequence, a replication origin sequence, a gene or coding sequence, hairpin loop sequences, and random sequences.

In panel 1B, the RNA template is converted to a double strand cDNA with a close loop structure at one end and a sticky end at the other end. To form a circle, a hairpin with a sticky end is ligated with the double strand DNA. The resulting circular cDNA is amplified by RCA. The hairpin DNA to be ligated to the double strand cDNA may further comprise a functional sequence (supra).

In panel 1C, the RNA template is converted to an RNA-DNA duplex with LoxP sites at both 3′ end and 5′ end. Cre-recombinase will circularize the duplex; RNase H will nick the duplex; and the RNA fragments may be used as primers for RCA. The resulting cDNA comprises 3′ and 5′ ends, which may further comprise a functional sequence (supra).

In panel 1D, the RNA template is converted to single strand cDNA with hairpins at both the 3′ and 5′ ends. The two hairpins are adjacent and ligated to form a circle. The resulting circular cDNA is amplified using RCA. The hairpins may further comprise a functional sequence (supra).

In panel 1E, the RNA template is converted to double strand cDNA with sticky ends at both ends. Ligating the 3′ and 5′ end sticky ends forms double strand circles (panel 1F). Alternatively, hairpins with sticky ends can be ligated with the double strand cDNA to form a circle (panel 1G). The resulting circular cDNA is amplified by using RCA. The hairpins of panels 1E, 1F and 1G, those to be ligated to the double strand cDNA, and those at both the 3′ and 5′ ends of the double strand cDNA, may further comprise a functional sequence (supra).

FIG. 2 shows several invention embodiments for circularizing and amplifying RNA using RCA. In panel 2A, a hairpin primer with polyT at the 3′ end is used to synthesize first strand cDNA. An oligo switch hairpin primer can be added to the 3′ end of the first strand cDNA by ligation, or polymerization and ligation. RNaseH will digest the RNA DNA duplex. The second strand cDNA is then synthesized and ligated to form a circle. The hairpin primers may further comprise a functional sequence (supra).

In panel 2B, a hairpin primer with polyT at the 3′ end is used to synthesize first strand cDNA. Self-priming is used to synthesize the second strand cDNA. Ligation 3′ end and 5′ end will form a circle. The hairpin primer may further comprise a functional (supra).

In panel 2C, a polyT primer with restriction enzyme site sequence will be used to synthesize the first strand cDNA, and self-priming is used to synthesize the second strand cDNA. Restriction enzymes cut the double strand cDNA to create a sticky end. A hairpin with a sticky end is then ligated with the double strand cDNA to form a circle. The poly-T primer or the hairpin to be ligated to the double strand cDNA may further comprise a functional sequence (supra).

In panel 2D, a polyT primer with a LoxP site sequence and an oligo switch primer with LoxP sequence is used to synthesize first strand cDNA. The resulting RNA DNA duplex is ligated with Cre-recombinase to form a circle. Rnase H is used to nick the RNA, and the resulting RNA fragments are used as primers to carry out the RCA. The poly-T primer and oligo switch primer may further comprise a functional sequence (supra).

FIG. 3 shows additional invention embodiments for circularizing and amplifying RNA and DNA using RCA. In panel 3A, the primer for synthesizing first strand cDNA contains a polyT sequence, a hairpin structure at the 5′ end, and an additional sequence identical to a segment of oligo switch primer. The polyT and oligo switch primers are used to synthesize first strand cDNA. Digestion and purification are used to eliminate the RNA template and unreacted oligo switch primer. The first strand cDNA will form two adjacent hairpins at both 3′ and 5′ ends, which can be ligated to form a circle. The poly-T primer and oligo switch primer may further comprise a functional sequence (supra).

In panel 3B, the primer for synthesizing first strand cDNA contains a polyT sequence and a restriction site at the 5′ end. The oligo switch primer similarly contains a restriction site. The polyT and oligo switch primers are used to synthesize first strand cDNA. The resulting RNA DNA duplex is cut with restriction enzyme, and the resultant sticky ends ligated to form a circle. Alternatively, prior to circularization, RNaseH digestion may be used to eliminate the RNA template, and a second round of DNA polymerization used to generate double stranded DNA, to be circularized as above. The poly-T primer and oligo switch primer may further comprise a functional sequence (supra).

In panel 3C, to amplify single strand or double strand cDNA, two hairpins are ligated to the 3′ and 5′ ends. The circular DNA is generated by extension of the 3′ end hairpin and ligation with the 5′ end hairpin. The resulting circular DNA can be amplified with RCA. The hairpin DNA at the 3′ and 5′ ends may further comprise a functional sequence (supra).

In panel 3D, to amplify double strand DNA, both ends of the double strand DNA were further extended by adding two short fragments as templates to hybridize to both ends for polymerase extension. The extended 3′ ends are complementary to each other; therefore the double strand DNA can be circularized. Alternatively, two hairpins with sticky ends can be ligated to the extended double strand DNA to create a circular DNA. The resulting circular DNA can be amplified with RCA.

FIG. 4 summarizes invention embodiments using RCA for SNP detection and amplifying a specific gene segment. Panel 4A shows a SNP site in the RNA template with regions A′ and B before and after the SNP site. A probe contains a region complementary to region A′ and a region identical to region B. The rest is arbitrary nucleotide sequence. The 3′ end of the probe is a base right of the SNP position. If the last base is matched with the SNP, the probe will be extended and create a complementary sequence region with probe region B. The extension will stop at a desired region by using a stop oligo to hybridize to the target RNA to create a RNA DNA duplex. If the last base is not matched with the SNP, the probe will not be extended, and a complementary sequence region with probe region B will not be created. Once the probe is extended, a hairpin structure is created. The resulting cDNA can be circularized with methods such as shown in FIGS. 2-3. The circular cDNA can be amplified with RCA. If the probe is not extended or extended non-specifically, the resulting cDNA will not be circularized and will not be amplified with RCA.

Panel 4B shows a SNP site in the target RNA with regions A′ and B′ before and after the SNP site, and additional regions C′ and D′ downstream from the B′ region. There may or may not be additional sequence regions in the target RNA between region B′ and C′. A probe contains a region A complementary to target RNA region A′, a region B′ identical to target RNA region B′, and additional pre-selected arbitrary sequence. The 3′ end of the probe is a base right of the SNP position. If the last base is matched with the SNP, the probe will be extended to create a complementary sequence region with probe region B′. The extension will stop at a desired region by using a stop hairpin oligo to hybridize to the target RNA to create a RNA DNA duplex. The stop hairpin oligo contains complementary region D and D′, a pre-selected arbitrary loop nucleotide sequence, and an additional region C′. The stop hairpin oligo hybridizes to the RNA target by controlling TM. If the probe last base is not matched with the SNP, the probe will not be extended, and a complementary sequence region with probe region B′ will not be created. Therefore the probe will not generate a hairpin structure after the target RNA is digested. Once the probe is extended correctly, the probe will be able to generate a hairpin structure after the target RNA is digested. The probe hairpin formed after extension has a sticky end complementary to the sticky end of the stop hairpin oligo. Therefore, the probe hairpin formed and stop hairpin oligo can be ligated to form a circle. The resulting circular nucleic acid molecule can carry out RCA. If the probe is not extended or extended non-specifically, the probe hairpin will not form and circularize with the stop hairpin oligo and will not be amplified with RCA. If there is one or more additional sequence regions in the target RNA between region B′ and C′, the extension C′ end of stop hairpin oligo is needed to circularize with the formed probe hairpin oligo after the target RNA is digested.

Panel 4C shows a SNP site in the target RNA with regions A′ and B′ before and after the SNP site, and additional region C′ and D′ downstream from the B′ region. A probe contains a region A complementary to target RNA region A′, a region B′ identical to target RNA region B′, a region C′ identical to target RNA region C′, and additional pre-selected arbitrary sequence. The 3′ end of the probe is a base right of the SNP position. If the last base is matched with the SNP, the probe will be extended and create a complementary sequence region with probe region B′. The extension will stop at a desired region by using a stop hairpin oligo to hybridize to the target RNA to create an RNA DNA duplex. The stop hairpin oligo contains complementary regions D and D′, a arbitrary loop nucleotide sequence, and an additional region C. The stop hairpin oligo hybridizes to the RNA target by controlling TM. If the probe last base is not matched with the SNP, the probe will not be extended, and a complementary sequence region with probe region B′ will not be created, and the probe will not generate a hairpin structure after the target RNA is digested. Once the probe is extended correctly, the probe will be able to generate a hairpin structure after the target RNA is digested. The probe hairpin formed after extension has a sticky end complementary to the sticky end of the stop hairpin oligo. Therefore, the probe hairpin formed and stop hairpin oligo can be ligated to form a circle. The resulting circular nucleic acid molecule can carry out RCA. If the probe is not extended or extended non-specifically, the probe hairpin will not form and circularize with the stop hairpin oligo, and will not be amplified with RCA.

Panel 4D shows a DNA template with region A down stream from the SNP site. The SNP probe at the 5′ end contains a sequence identical to region A. The 3′ end of the SNP probe is a base right in the SNP position. If the last base is matched with the SNP, the probe will be extended and create a complementary sequence, which will hybridize with the 5′ end of the SNP probe sequence to form a hairpin structure. If the last base is not matched with the SNP, the probe will not be extended, and a hairpin structure will not be created. An additional probe up stream of the SNP probe is a strand displacement probe, which will displace the SNP probe from the template. Once the SNP probe is displaced from the template after extension, it can be circularized with the method shown in panel 4A. The resulting circular DNA can be amplified with RCA to differentiate the SNP.

FIG. 5 summarizes detection of RNA and DNA with circular probes. In panel 5A, a single strand full length gene is generated and circularized from a full length cDNA clone library or from RNA. The circle may also contain pre-selected arbitrary nucleotide sequence. The resulting circular DNA can be used as a probe to detect target RNA from a complex mixture. The RNA to be detected will hybridize with the circular DNA and be nicked with Rnase H. The nicked target RNA fragment can be used as primers for RCA. To avoid the poly-A RNA tail from functioning as a primer, the full-length single strand circular DNA will have a poly-A portion instead of poly-T portion. Once the target RNA has bound to the circular DNA and RCA has begun, the poly-A tails of the mRNA will bind to RCA product with the complementary poly-T portion as primers for further amplification. In such case, exponential amplification is achieved without adding any primers to the reaction system.

In panel 5B, the full-length circular gene can hybridize with first strand cDNA, and the first strand cDNA will be used as primers for RCA. The full-length circular DNA may further comprise a functional sequence (supra).

In panel 5C, a circular DNA is used as a probe and added to a complex mixture for RNA target detection. The target RNA is treated before or after the hybridization to the circular probes to create 3′ ends as primers for RCA. For instance, a circular DNA may contain a segment of O-methyl-RNA. Once the O-methyl-RNA DNA circular probe is hybridized with RNA target, the targeted RNA-DNA duplex will be digested by Rnase H, but not the O-methyl RNA-RNA duplex. The nicked targeted RNA can be used as primer for RCA. Additional primers can be added to carry out exponential amplification. The circular DNA may further comprise a functional sequence (supra).

Panel 5D shows the circular DNA used for DNA detection. The probes of a RNA fragment, DNA fragment or a chimeric DNA-RNA fragment will not hybridize with the circular DNA as primers to initiate the RCA until it is hybridized with targeted DNA to be nicked with Rnase H. In one embodiment, one probe is a DNA hairpin, and the other probe is an RNA DNA chimeric hairpin. Once the two hairpins are hybridized to the targeted DNA at adjacent position, the RNA-DNA duplex will be formed. Rnase H will nick the RNA DNA duplex to create a primer for RCA, which indicates the presence of the target DNA. Once the RNA DNA duplix is nicked, the remaining RNA DNA chimeric hairpin fragment will be replaced by a new RNA DNA chimeric hairpin to form a new RNA DNA duplex. The process will be cycled, generating amplification on top of amplification by RNA. The circular DNA may further comprise a functional sequence (supra).

Panel 5E shows circular DNA used for RNA detection. The probes of an RNA fragment, a DNA fragment, or a chimeric DNA-RNA fragment will not hybridize with the circular DNA as primers to initiate the RCA until it is hybridized with targeted RNA to be nicked with Rnase H. In one embodiment, one probe is a hairpin with a DNA complementary region and a loop RNA region, and the other probe is an RNA DNA chimeric hairpin with complementary RNA region, a loop RNA region, and an additional DNA fragment. Once the two hairpins are hybridized to the targeted RNA at adjacent position, the RNA-DNA duplex will be formed. Rnase H will nick the RNA DNA duplex to create a primer for RCA, which indicates the presence of the target DNA. Once the RNA DNA duplex is nicked, one of the remaining RNA DNA chimeric hairpin fragments will be replaced by a new RNA DNA chimeric hairpin to form a new RNA DNA duplex. The process will be cycled to generate amplification on top of amplification by RCA. The circular DNA may further comprise a functional sequence (supra).

Panel 5F shows circular DNA used for both RNA and DNA detection and amplification. Two probes will be introduced into a complex mixture for RNA DNA detection. One probe is pre-circularized DNA, and the other probe is a DNA hairpin.

The circular probe contains three regions: one region is complementary to detection target; the second region is complementary to the arm of hairpin DNA probe; and the third region is pre-selected arbitrary nucleotide sequence. The hairpin DNA also contains three regions: the loop region is complementary to the detection target region, which is adjacent to the circular probe target region; the arm region is complementary to part of circular probe; and the optional third region is pre-selected arbitrary nucleotide sequence. If the target is not present, the circular probe will not hybridize with the arm of DNA hairpin to initiate RCA. However, if the target is present, both the circular probe and hairpin probe will hybridize to the target template at adjacent positions. In the same time, the hairpin probe will also hybridize with the circular probe to become a primer to initiate RCA. The RCA reaction will detach the circular probe from its hybridization with the target. If an additional primer is used for exponential amplification by RCA, the primer will hybridize with amplified RCA products to detach the hairpin probe from target. Therefore released target can be used for second and subsequent round hybridizations with circular and hairpin probes to initiate RCA. The process will be cycled to generate exponential amplification on top of the amplification by RCA.

The following examples are included to demonstrate various embodiments of the invention. It should be appreciated by those of skill in the art that the techniques disclosed in the examples are representative embodiments of the invention, but the invention is not limited to the examples presented herein. Those of skill in the art will, in light of the present disclosure, appreciate that many alternative embodiments to those disclosed herein exist and will yield similar results without departing from the spirit and scope of the invention.

### EXAMPLES

### Example 1

### Synthesis of the cDNA with Complementary Ends

MMLV reverse transcriptase (RT) has the ability to add cytosine residues to the 3′ end of newly synthesized cDNAs upon reaching 5′-end of the mRNA template. Usually 2-4 cytosine residues are added, depending on the reaction conditions.

mRNA is purified using standard methods that prevent RNA degradation. Small amounts of mRNA, as low as picrogram amounts, are used as the target nucleic acid molecule. A first strand synthesis primer containing poly(dT) and a T7 transcriptional promoter at its 5′ end, primer 1, and MMLV reverse-transcriptase enzyme are added to the mRNA sample. The poly(dT) sequence of the first strand synthesis primer anneals to the poly(A) tail of mRNA, serving as a primer for reverse-transcriptase to synthesize first strand cDNA. Simultaneously, primer 2 anneals to primer 1. At the 3′ end of the first strand cDNA, reverse-transcriptase adds a few cytosine residues. The 5′ end of first strand cDNA has the T7 promoter followed by a poly(T) stretch, as this sequence was used as the primer for the first strand synthesis. The T7 promoter is oriented such that once the molecule is circularized the promoter will direct transcription of a copy of the original mRNA.
Primer 1: 5′-d(T7 promoter sequence)+d(T) 15-3′
Primer 2: 5′-d(T7 promoter sequence complement)+d(G)4-3′

10 pmol of cDNA synthesis primers are annealed to 1. μg of human placenta poly(A)⁺ RNA (Clontech), in a volume of 5 μL of deionized water, by heating the mixture for 2 minutes at 70° C., followed by cooling on ice for 2 minutes. First-strand cDNA synthesis is then initiated by mixing the annealed primer-RNA complexes with 200 units of M-MLV RNaseH-reverse transcriptase (superScript II reverse transcriptase, Life Technology) in a final volume of 10 μl containing 50 mM Tris-HCl (pH 8.3 at 22° C.); 75 mM KCl; 6 mM MgCl₂; 1 mM DTT; and 1 mM each of dATP, d GTP, dCTP, and dTTP.

To the above reaction solution, 1 u of RNAse H is added and incubated for 1.5 hours. The resulting solution is purified with Qiagen kit and then detected with UV absorbance to measure the amount of cDNA with Nanodrop instruments. The OD indicated about 140 ng of cDNA is obtained. The resultant nucleic acid will have a 3′ overhang of d(C) on one end and a 3′ overhang of d(G) on the other end. The overhangs may anneal and allow template switching thus generating a circular molecule. Ligase may be added to link the ends of the molecules.

### Example 2

### Synthesis of the cDNA with LoxP Recombination Sites

A LoxP recombination site may be added by the oligo switch technology. An oligonucleotide with oligo(G) or oligo(rG) sequences at its 3′ most end is included in the first strand cDNA synthesis medium. Its terminal 3-4 G residues will base pair with the 2-4 C residues of the newly synthesized cDNA, thus serving as a new template for the RT (template switch). The RT then switches the template and replicates the sequence of the oligo(G) oligonucelotide, thus including the complementary CapFinder oligonucleotide sequence at the 3′ end of the newly synthesized cDNA.
Primer 3: 5′-d(LoxP sequence)+d(T)15-3′
Primer 4: (sequence for oligo switch) 5′-d(LoxP sequence)r(GGGp)-3′

10 pmol of cDNA synthesis primer 3 are annealed to 1 μg of human placenta poly(A)⁺ RNA (Clontech), in a volume of 5 μL of deionized water, by heating the mixture for 2 minutes at 70° C., followed by cooling on ice for 2 minutes. First-strand cDNA synthesis is then initiated by mixing the annealed primer-RNA complex with 200 units of M-MLV Rnase H-reverse transcriptase (superScript II reverse transcriptase, Life Technology) in a final volume of 10 μl containing 50 mM Tris-HCl (pH 8.3 at 22° C.); 75 mM KCl; 6 mM MgCl₂; 1 mM DTT; and 1 mM each of dATP, d GTP, dCTP, and dTTP. The first-strand cDNA synthesis-template switching reaction is incubated at 42° C. for 1.5 hours in an air incubator and then cooled on ice.

To the above reaction solution, 1 u of RNAse H is added and incubated for 1.5 hours. The resulting solution is purified with Qiagen kit and then detected with absorbance to measure the amount of cDNA with Nanodrop instruments. The OD indicated about 160 ng of cDNA is obtained.

### Example 3

### An Alternative Method: Use Terminal Transferase Enzyme to Add Homologous Sequences to the 3′ End of the First Strand cDNA

The synthesized first strand cDNA is purified with Qiagen kit. Then the first strand 0.5 ug cDNA is mixed with 0.5 uM dCTP, 1×Reaction buffer of Terminal Transferase and 1 unit of Terminal transferase (Finnzymes) at 37 degree for 1.5 hours. The resulting solution is purified with Qiagen kit and detected with Bioanalyer (Agilent). It is finally quantified with Nanodrop absorbance indicating 0.45 ug of cDNA.

### Example 4

### Synthesis of the Circular Molecule

The first strand cDNA can be circularized with ligation by using a short oligonucleotides as a bridge. After the first strand cDNA is synthesized, an oligonucleotide with sequences complementary to the sequences at both 3′ end and 5′ end of the newly synthesized cDNA is incubated with T4 DNA ligase in the incubation medium. The resulting cDNA will be circularized.
Primer 5: 5′-d(Complementary sequence of T7)+dGdGdG-3′

Incubate the first strand cDNA with T4 DNA ligase (Promega) in 1×T4 DNA ligation buffer (Promega), 0.5 mM ATP and primer 5 at room temperature overnight. Then 0.5 U Exonuclease V (Amersham) and 0.5 mM ATP are added to the above solution for another 1.5 hours. All the linear strand DNA is digested. The resulting circular cDNAs is purified with Qiagen kit and measured with absorbance. 0.5 ug circular cDNA is produced.

### Example 5

### Circularization by Randomer Hybridization

The invention may be used to amplify double stranded target nucleic acid molecules. The following example illustrates a method of amplifying an entire target nucleic acid without reference to the sequence of the target. As such, the method could be adapted for amplification of entire genomes or other large samples of double stranded nucleic acid molecules.

Total genomic DNA is digested with Pml I in 10 mM Bis Tris Propane-HCl, 10 mM MgCl₂, 1 mM dithiothreitol (pH 7.0@25° C.), 100 μg/ml BSA, 100 μM dNTPs by incubating at 37° C. for one hour.

T4 DNA polymerase is added to the mix with excess of a linker oligonucleotide containing in the 5′ half a LoxP site for CreI dependent recombination and a random hexamer sequence at the 3′ end. The reaction is incubated overnight at 37° C. The resulting products will be genomic fragments with a LoxP site at either end of the nucleic acid.

CreI is then added to the sample and incubated at 37° C. for one hour. The resulting products are circularized fragments of the entire genome suitable for rolling circle amplification.

### Example 6

### Circularization by Recombination

The first strand cDNA can be circularized with Cre-recombinase. The first strand cDNA is synthesized with loxP sites at both 3′ end and 5′ end as described in Example 2. Incubation the first strand cDNA with CreI recominbinase in an incubation medium will circularize the first strand cDNA.

The first strand cDNAs synthesized with LoxP sequences at both the 3′ end and the 5′ end is incubated with Cre-recombinase at 37 degree for 4 hours. The recombinase is deactivated by increasing the temperature to 75° C. for 30 min. Then 0.5 U Exonuclease V (Amersham) and 0.5 mM ATP are added to the above solution and incubated for 1.5 hours. The circularized cDNA is purified with Qiagen kit and measured with absorbance (0.6 ug).

### Example 7

### Amplification of the Circular cDNAs

The circularized cDNA can be amplified with rolling circle amplification by performing a limited RNaseH digest of an mRNA:first strand cDNA complex. The resulting nicked mRNA can be used as primers to perform RCA amplification by virtue of the free 3′ ends. This method will yield free 3′ ends in only one strand as is required for RCA.

The circularized cDNAs with RNA:DNA duplex is incubated with 0.5 U RNase H at 37 degree for 30 minutes. Then 4 μL of the above reaction is incubated in a volume of 35 μL containing 20 mM Tris.HCl (pH=8.8), 10 mM KCl, 2.7 mM MgSO4, 5% v/v DMSO, 0.1% Triton X-100, 400 μM dATP, dGTP, dCTP, dTTP and 900 nM of Primer 5 (a primer for exponential amplification; see, U.S. Ser. No. 60/506,218). Phage T4 gene-32 protein (Amersham) is present at a concentration of 38 ng/uL, (approximately 1085 nM). After combining all the materials at RT, the reactions are placed on ice, Vent (exo_DNA polymerase (New England Biolabs) is added at a final concentration of 0.32 units/ul, and the reactions are incubated at 75 degree for 3 min, then at 65.5 degree for 90 mins. The resulting mixture is run on a gel. The rolling circle products are observed at the top of the gel after staining, due to not having entered the gel.

### Example 8

### Amplification of cDNAs with Random Priming

The circularized cDNA can be amplified with rolling circle amplification by using random hexamer as primers. The random hexamers will only amplify circular nucleic acid molecules. RNA digestion or heat denaturation is used to disassociate the mRNA from the first strand circular cDNA. Heat denaturation may also be used to dissociate dsDNA in preparation for amplification. The ssDNA may then be isolated for use as a reagent in other biological applications. To the ssDNA, random hexamer can be added along with a strand displacement polymerase such as Phage 29, vent and BST. The mixture would be incubated at the appropriate temperature for the strand displacement polymerase for the desired period of time.

4 uL of single strand circularized DNA is incubated in a volume of 35 uL containing 20 mM Tris.HCl (pH=8.8), 10 mM KCl, 2.7 mM MgSO4, 5% v/v DMSO, 0.1% Triton X-100, 400 uM dATP, dGTP, dCTP, dTTP and 900 nM of the Random Hexamer (Amersham). Phage T4 gene-32 protein (Amersham) is present at a concentration of 38 ng/uL, (approximately 1085 nM). After combining all these materials at RT, the reactions are placed on ice. Vent (exo-) DNA polymerase (New England Biolabs) is added to a final concentration of 0.32 units/ul, and the reactions are incubated at 75° C. for 3 minutes, then at 65° C. for 90 minutes. The resulting mixture is run on a gel. The rolling circle products are observed at the top of the gel after staining, due to not having entered the gel.

### Example 9

### In Vitro RNA Transcription

In vitro RNA transcription may be conducted using any of the above circular cDNA as a template. With the addition of T7 polymerase and rNTPs, T7 polymerase will transcribe either the sense or antisense strand of the cDNA depending upon the selected orientation of the T7 promoter.

The resulting double strand RCA products are transcribed with T7 RNA polymerase. 3 ng of cDNA is transcribed in each reaction. Reactions conditions are: 40 mM Tris pH 7.5, 6 mM MgCl₂, 10 mM NaCl, 2 mM spermidine, 10 mM DTT, 500 μM each ATP, GTP, and UTP-cy3, 12.5 μM CTP, 10 units Rnase block, and 80 units T7 RNA polymerase in a volume of 20 μl. Reactions are incubated at 37° C. for 2 hour. The resulting mixture is purified with a Qiagen kit. The synthesized dye labeled aRNA is eluted with ethanol and measured with Nanodrop.

### Example 10

### Circularization of the Detection Circle

A linear full-length GAPDH sequence (Integrated DNA Technologies, Skokie, Ill.) with the 5′ end phosphorylated is circularized with a template sequence; e.g. U.S. Ser. No. 60/506,218. Nucleic acid sequences to amplify and detect GAPDH are described in our U.S. Ser. No. 60/506,218. The circularization reaction contains 50 μM circle precursor, 50 μM template, 100 mM NiCl₂, 200 mM imidazole.HCl (pH=7.0), and 125 mM BrCN, and the reaction is allowed to proceed 10 h at 23° C. After dialysis and lyophilization the product is purified by preparative denaturing 20% polyacrylamide gel electrophoresis, and the product band is isolated by excision, crushing, and eluting into 0.2 M NaCl. The salts are removed by dialysis against distilled deionized water, and the DNA is quantitated by absorbance at 260 nm, using the nearest neighbor method to calculate molar extinction coefficients.

### Example 11

### mRNA Amplification and Detection

Total RNA is obtained from Clontech. The total RNA is pre-processed with a ribozyme that cleaves the GAPDH mRNA at a 3′ end sequence; U.S. Ser. No. 60/506,218. 0.5 μg of processed total RNA is mixed with the circular oligonucleotide prepared in Example 9 in 20 mM Tris. HCl (pH=8.8), 10 mM KCl, 2.7 mM MgSO4, 5% v/v DMSO, 0.1% Triton X-100, 400 μM dATP, dGTP, dCTP, dTTP. The mixture is heated to 75° C. for 5 minutes. Then the mixture is cooled to room temperature slowly. To the above reaction are added 1 U phage 29 (Amersham), 1 U RNaseH and Phage T4 gene-32 protein (Amersham) with a concentration of 38 ng/ul. The resulting mixture is incubated at 37° C. for 4 hours. Then the reaction mixture is incubated at 95° C. for 10 min to deactivate all the enzymes. The double strand cDNA is precipitated out with phenol chloroform. The precipitated DNA is run on a gel and stained. Gel staining shows the long double strand cDNA located at the top of the wells. Detection can be performed by any of the methods available to one of skill in the art. To enhance amplification, an amplification primer (U.S. Ser. No. 60/506,218) may be added to the above reaction. Addition of this primer will result in (n!) factorial amplification.

### Example 12

### Multiplexed Detection Reaction

For detection of multiple genes in a single tube, the same reaction as described in Example 11 can be carried out by combining multiple gene specific or mRNA specific circular templates in the same reaction.

The invention may be implemented as methods and processes, and also as kits comprising recited reagents and compositions for practicing recited methods, and business methods which comprise implementing, selling, teaching, demonstrating and/or marketing the foregoing methods and compositions.

It is understood that the examples and embodiments described herein are for illustrative purposes only and that various modifications or changes in light thereof will be suggested to persons skilled in the art and are to be included within the spirit and purview of this application. All publications, patents and patent applications cited herein are hereby incorporated by reference in their entirety for all purposes to the same extent as if each individual publication, patent or patent application were specifically and individually indicated to be so incorporated by reference.

1-53. (Canceled)

54. A method of amplifying a polynucleotide, comprising:
a) forming a linear polynucleotide having 3′ and 5′ hairpins;
b) ligating 3′ and 5′ ends of the linear target to form a circularized polynucleotide; and
c) amplifying the circularized polynucleotide by rolling circle amplification.

55. The method of claim 54, further comprising prior to the forming step:
a) hybridizing a first hairpin to a 3′ portion of a target nucleic acid; then b) polymerizing copy DNA from the 3′ end of the first hairpin using the target as a template; and
c) affixing a second hairpin to a 5′ portion of the target or a 3′ portion of the copy DNA to form the linear polynucleotide.

56. The method of claim 55, wherein the affixing step is effected by self-priming.

57. The method of claim 56, wherein the affixing step is effected by self-priming using a template switching oligonucleotide to extend the target nucleic acid.

58. The method of claim 57, wherein the template switching oligonucleotide comprises a restriction site, the first hairpin is formed by cutting double-strand DNA of target DNA and template switch oligonucleotide at the restriction site to form a cut end, and ligating to the cut end a corresponding cut end of a hairpin.

59. A method according to claim 54 for making circular copy DNA by:
a) hybridizing a first primer to a 3′ portion of a template region of a target strand;
b) polymerizing from the primer a first copy DNA of the template region;
c) displacing from the template region the first copy DNA;
d) forming a hairpin second primer at a 3′ portion of the first copy DNA;
e) polymerizing from the hairpin primer a second copy DNA of a portion of the first copy DNA; and
f) ligating the 5′ end of the first copy DNA with the 3′ end of the second copy DNA to form a circular copy DNA.

60. A method according to claim 59, wherein the displacing step is effected with nuclease, base or strand displacement.

61. A method of amplifying a DNA comprising:
a) polymerizing a copy DNA of a template region of a DNA target to form a double-stranded DNA having first and second hairpins at first and second ends, respectively;
b) ligating the double-stranded DNA in a single molecular reaction to form a circularized DNA; and
c) amplifying the circularized DNA by rolling circle amplification.

62. The method of claim 61, wherein the target is transcribed from an mRNA

63. The method of claim 61, wherein the target is transcribed from an mRNA and the first hairpin is formed by self-priming of the target.

64. The method of claim 61, wherein the target is transcribed from an mRNA with at least one template switching hairpin oligonucleotide, the first hairpin is formed by covalently attaching said template switching hairpin oligonucleotide to the 3′ end of the target, optionally with the template switching hairpin oligonucleotide serving as a template for extension of the 3′ end of the target.

65. The method of claim 61, wherein the target is transcribed from an mRNA, the second hairpin is formed by initiating polymerization of the target with a hairpin primer, wherein the hairpin is formed before or after polymerization of the target.

66. The method of claim 61, wherein the target is transcribed from an mRNA using a primer comprising a restriction site to initiate polymerization of the target, the second hairpin is formed by cutting the double-stranded DNA at the restriction site to form a cut end, and ligating to the cut end a corresponding cut end of a hairpin.

67. The method of claim 61, wherein the target is genomic DNA, wherein the first and second hairpins are formed by ligating first and second ends of the genomic DNA to corresponding cut ends of corresponding hairpins.

68. The method of claim 61, wherein the target is a single stranded DNA copied from a genomic DNA or cDNA library or transcribed from an mRNA, the first and second hairpins are formed by self-hybridization of 3′ and 5′ ends of the target DNA at adjacent positions, and the ligating step comprises covalently linking the 3′ and 5′ ends to form the circularized DNA.

69. The method of claim 61, wherein the target is transcribed from an mRNA with a template switching oligonucleotide comprising at its 3′ end at least one nucleotide which basepairs with a nucleotide at the 3′ end of the target strand of an RNA-DNA intermediate comprising the mRNA and the target, wherein the template switching oligonucleotide serves as a template for extension of the target, and has a pre-selected arbitrary nucleotide sequence at its 5′ end, and extending the 3′ end of the target to produce a DNA that is complementary to the mRNA molecule and the template switching oligonucleotide.

70. A method of amplifying a linear nucleic acid target, comprising steps:
a) affixing a first oligonucleotide linker comprising a hairpin to one end of the target;
b) forming a hairpin at the other end of the target;
b) circularizing the target;
c) generating a free 3′ end on the target; and
d) amplifying the target from the free 3′ end by rolling circle amplification.

71. The method of claim 70 wherein the circularizing step is performed by a method selected from the group consisting of annealing complementary ends followed by ligation, and self-priming followed by ligation.

72. The method of claim 70 wherein the target nucleic acid molecule is a first strand cDNA.

73. The method of claim 70 wherein the first linker is affixed by hybridization and ligation, hybridization followed by polymerase extension, enzymatic reaction, chemical reaction, and photo-reaction.

74. The method of claim 70 further comprising affixing a second oligonucleotide linker prior to circularization.

75. The method of claim 74 wherein the second linker is affixed by a method selected from the group consisting of ligation, hybridization and ligation, hybridization followed by polymerase extension, self-priming and ligation, enzymatic reaction, chemical reaction, and photo-reaction.

76. The method of claim 70 wherein the first linker is affixed by randomer hybridization and extension from nucleic acid breath.

77. The method of claim 70 wherein the generating step comprises adding one or more primers comprising the free 3′ end.

78. The method of claim 70 wherein the generating step comprises nicking a strand of target, wherein the target is double-stranded.

79. The method of claim 77 wherein the one or more primers further comprise random sequences at there 3′ ends.

80. The method of claim 77 wherein the one or more primers further comprise promoter sequences.

81. The method of claim 77 where the one or more primers are RNA:DNA chimeras.

82. The method of claim 70, wherein the circularized target comprises a sequence for in vitro or vivo protein expression.
